(19) United States
(12) Patent Application Publication
(10) Pub. No.: US 2008/0260674 A1
(43) Pub. Date: Oct. 23, 2008

(54) Cosmetic Use of Amphoteric Polysaccharide Compounds Containing Cationic Polymer Chain (S)

(76) Inventor: Michel Philippe, Wissous (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/660,381

(22) PCT Filed: Aug. 18, 2005
(86) PCT No.: PCT/EP05/09985

§ 371 Date: Nov. 13, 2007

Related U.S. Application Data
(60) Provisional application No. 60/612,170, filed on Sep. 23, 2004.

(30) Foreign Application Priority Data
Aug. 19, 2004 (FR) 0408997

(51) Int. Cl. A61K 8/91; A61K 8/73; A61Q 5/00; A61Q 19/00

(52) U.S. Cl. 424/70.13; 424/78.17; 514/772.1

(57) ABSTRACT

The invention relates to the cosmetic use, especially for the cosmetic treatment of keratin materials, of amphoteric polysaccharide compounds containing cationic polymer chain(s), which may be obtained by grafting and polymerization of ethylenic monomers of formula (I), Q⁻ onto an anionic polysaccharide of formula (II): in the presence of a catalytic system based on potassium permanganate (KMnO₄) and sulfuric acid (H₂SO₄). The invention also relates to compositions comprising at least one cosmetic active agent and at least one of these amphoteric polysaccharide compounds, in a cosmetically acceptable medium.

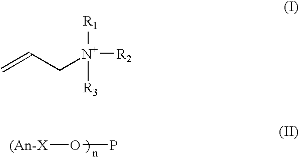

The present invention relates to the use of amphoteric polysaccharide compounds containing cationic polymer chain(s) in cosmetics, and to compositions comprising them.

In the cosmetics field, it is especially sought to improve the cosmetic properties of keratin materials, such as the hair and the skin, and more particularly sensitized hair, i.e. hair that has become damaged or embrittled, especially due to the chemical action of atmospheric agents and/or of hair treatments such as permanent-waving, dyeing or bleaching.

With this aim, it is common practice to use complementary cosmetic agents known as conditioning agents, for example cationic polymers or silicones, which are intended mainly to repair or limit the harmful or undesirable effects induced by the various treatments or attacking factors to which hair fibres are more or less repeatedly subjected. These conditioning agents also improve the cosmetic behaviour of natural hair.

Other conditioning agents, such as the amphoteric polysaccharides described in documents U.S. Pat. No. 4,803,071, U.S. Pat. No. 4,464,523, WO 90/03779 and FR 2 883 599 may be used in cosmetic hair compositions. However, these polysaccharides are not very efficient as regards conditioning and remanence.

The Applicant has found that, after several uses, the hair becomes laden and lacks lightness.

The Applicant has thus found, surprisingly and unexpectedly, that the use of amphoteric polysaccharide compounds containing cationic polymer chain(s), in cosmetics, makes it possible to overcome the drawbacks described above and to obtain excellent cosmetic properties such as an excellent conditioning and protecting effect on the hair, good disentangling of the hair and good hold of the hairstyle. In addition, the use of these amphoteric polysaccharide compounds containing cationic polymer chain(s) leads to good remanence of these properties, even after washing the hair several times, without observing an excessive deposit that would lead to laden, non-malleable and non-supple hair.

These conditioning agents also give the skin cosmetic properties such as good moisturization.

These amphoteric polysaccharide compounds containing cationic polymer chain(s) may be obtained according to the preparation process as described in the articles “Modification of sodium carboxymethyl-cellulose by grafting of dimethyldiallylammonium chloride” by Li-Ming Zhang, Macromol. Mater. Eng., 2000, 280/281, pages 66-70 and “Manganese (IV)-initiated grafting of trimethylallylammonium chloride onto carboxymethylcellulose” by Li-Ming Zhang, J. M. S.—Pure Appl. Chem., A36(9), 1999, pages 1141-1152.

This process makes it possible to obtain pure polysaccharides, i.e. polysaccharides containing no trace of homopolymer.

One subject of the present invention is thus the use of amphoteric polysaccharide compounds as described below in cosmetics and especially for the cosmetic treatment of keratin materials, such as caring for and protecting the hair, hairstyling, permanent-waving, relaxing, dyeing or bleaching the hair, or alternatively cleansing and care of the skin.

A subject of the invention is also a cosmetic composition comprising at least one cosmetic active agent and at least one polysaccharide compound as described below, in a cosmetically acceptable medium.

Other subjects, characteristics, aspects and advantages of the invention will emerge even more clearly on reading the description and the various examples that follow.

The amphoteric polysaccharide compounds containing cationic polymer chain(s) used according to the invention may be obtained by grafting and polymerization of ethylenic monomers corresponding to formula (I) below:

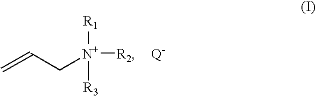

onto anionic polysaccharides of formula (II):

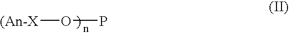

in the presence of a catalytic system based on potassium permanganate (KMnO₄) and sulfuric acid (H₂SO₄).
In formulae (I) and (II):
R₁, R₂ and R₃ represent, independently of each other, a hydrogen atom or a linear or branched, saturated or unsaturated C₁-C₂₂ and preferably C₁-C₁₈ monovalent hydrocarbon-based group,
Q represents a mineral or organic anion, for example a halogen atom, such as a bromine or chlorine atom, or an acetate, a citrate, a lactate, an oleate or a behenate, a halogen atom being preferred, and a chlorine atom being particularly preferred,
P represents a polysaccharide chain,
X represents a linear or branched, saturated or unsaturated, optionally hydroxylated C₁-C₁₈ divalent hydrocarbon-based group,
An represents an ionic group chosen from:

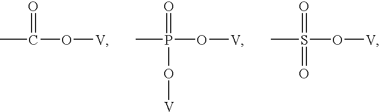

with V representing a hydrogen atom or an alkali metal or alkaline-earth metal, for example a sodium or potassium atom,
n is such that the degree of substitution of the polysaccharide compound with an anionic group (DS(−)), is between 0.02 and 2 and preferably between 0.05 and 1.5.

The term “degree of substitution DS(−) of the amphoteric polysaccharide compounds” according to the invention means the ratio of the number of hydroxyl groups substituted with an anionic group in the repeating unit to the number of elementary monosaccharides constituting the unit.

The polysaccharide chain represented by P is preferably a cellulose, a hemicellulose, a starch, an insulin, a guar gum, a xanthan gum, a pullulan, an agar-agar, a sodium, potassium or ammonium alginate, a carrageenan, a dextran, a furcellaran, a gellan gum, a gum arabic, a gum tragacanth, a hyaluronic acid, a konjac mannan, a lignin sulfonate, a carob gum, a partially N-acetylated chitin, a pectin, a polydextrose, a rhamsan gum or a welan gum.

More preferably, the polysaccharide chain is a cellulose, a hemicellulose, a hydroxyethylcellulose, a hydroxypropylcellulose, a hydroxypropylmethylcellulose, a methylcellulose, a starch, a starch acetate, a hydroxyethyl starch, a hydroxypropyl starch, an insulin, a guar gum, a hydroxyethylguar gum, a hydroxypropylguar gum or a xanthan gum.

The polysaccharide chain containing sulfonate function(s) preferably has a weight-average molecular mass of between 500 and 15 000 000 and better still between 1000 and 10 000 000.

Examples of linear or branched, C₁-C₂₂ and preferably C₁-C₁₈ monovalent hydrocarbon-based groups that may especially be mentioned include linear or branched C₁-C₄ alkyl groups, such as methyl, ethyl, n-propyl, isopropyl, n-butyl or tert-butyl groups, linear or branched C₁₂-C₁₈ alkyl groups, such as lauryl, myristyl, cetyl or stearyl groups, and linear or branched C₂-C₁₈ and preferably C₂-C₆ alkenyl groups such as vinyl, allyl, crotonyl or butenyl; the C₁-C₄ or C₁₂-C₁₈ alkyl groups being particularly preferred.

Examples of linear or branched, saturated or unsaturated C₁-C₁₈ and preferably C₁-C₁₂ divalent hydrocarbon-based groups that may especially be mentioned include linear or branched C₁-C₁₈ alkylene groups, such as methylene, ethylene, n-propylene, isopropylene, n-butylene, tert-butylene, hexylene or octylene groups; linear or branched C₂-C₁₈ alkylene groups, such as vinylene, allylene, crotonylene, isobutenylene, hexenylene or octenylene. These groups may also bear at least one hydroxyl substituent.

The amphoteric polysaccharide compounds containing cationic polymer chain(s) preferably used in the present invention are those that may be obtained by grafting and polymerization of ethylenic monomers corresponding to formula (I) below:

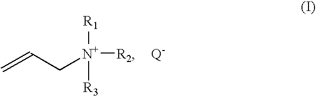

onto anionic polysaccharides of formula (II):

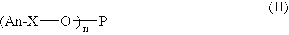

in the presence of a catalytic system based on potassium permanganate (KMnO₄) and sulfuric acid (H₂SO₄),
in which formulae (I) and (II):

R₁, R₂ and R₃ represent, independently of each other, a hydrogen atom or a C₁-C₄ or C₁₂-C₁₈ alkyl group,
Q represents a halogen atom,
P represents a polysaccharide chain chosen from a cellulose, a hemicellulose, a hydroxyethylcellulose, a hydroxypropyl-cellulose, a hydroxypropylmethylcellulose, a methylcellulose, a starch, a starch acetate, a hydroxyethyl starch, a hydroxypropyl starch, an insulin, a guar gum, a hydroxyethylguar gum, a hydroxypropylguar gum or a xanthan gum,
X represents an optionally hydroxylated methylene, ethylene, n-propylene, isopropylene, n-butylene, tert-butylene, hexylene or octylene, vinylene, allylene, crotonylene, isobutenylene, hexenylene or octenylene group, and
   An and n are as defined above.

The amphoteric polysaccharide compounds containing cationic polymer chain(s) that are the most particularly preferred are those obtained from a carboxymethylcellulose and trimethylallylammonium bromide or chloride or diallyldimethylammonium chloride.

The amphoteric polysaccharide compounds according to the invention may be prepared via the process described in the articles “Modification of sodium carboxymethylcellulose by grafting of dimethyldiallylammonium chloride” by Li-Ming Zhang, Macrom. Mater. Eng., 2000, 280/281, pages 66-70, and “Manganese (IV)-initiated grafting of trimethylallylammonium chloride onto carboxy-methylcellulose” by Li-Ming Zhang, Pure Appl. Chem., A36(9), 1999, pages 1141-1152.

An anionic polysaccharide of formula (II) is mixed with a KMnO₄ solution. Sulfuric acid and a quaternary ammonium halide of formula (I) are then added thereto in this order.

The amounts of the various compounds will be readily determined by a person skilled in the art depending on the desired degree of substitution with a cationic group. This degree of substitution with a cationic group is preferably within the range from 0.01 to 2 and better still from 0.05 to 1.5.

The total degree of substitution of the amphoteric polysaccharide compound containing cationic polymer chain(s) as described above is within the range preferably from 0.03 to 3 and better still from 0.05 to 2.5.

The amphoteric polysaccharide compounds as described above are used in cosmetics, and especially for the cosmetic treatment of keratin materials, such as caring for and protecting the hair, as a conditioning agent; for the hold and discipline of the hairstyle, as a fixing agent; but also for cleansing and caring for the skin and for making up the skin, the lips and the nails.

A subject of the present invention is also a cosmetic composition comprising, in a cosmetically acceptable medium, at least one cosmetic active agent and at least one amphoteric polysaccharide compound containing cationic polymer chain(s) as described above, preferably in an amount ranging from 0.05% to 50% by weight and better still from 0.5% to 25% by weight relative to the total weight of the composition.

For the purposes of the present invention, the term “cosmetic active agent” means an active agent that has beneficial effects on the skin, the hair and/or the nails.

The cosmetic active agent used in the composition according to the present invention may be a conditioning agent that is well known in the art, such as an anionic, cationic, nonionic, amphoteric or zwitterionic polymer, a mineral, plant or synthetic oil, a fatty acid ester, a volatile or non-volatile, organomodified or non-organomodified, cyclic or acyclic, branched or unbranched silicone; a dye, an anti-UV agent, a sunscreen, a hydroxy acid or a vitamin, which are well known in the art.

The term “cosmetically acceptable medium” means a medium that is compatible with any keratin material, such as the skin, the hair, the nails, the eyelashes, the eyebrows and the lips and any other area of body or facial skin.

The cosmetically acceptable medium may consist solely of water or of a mixture of water and of a cosmetically acceptable solvent such as a C₁-C₄ lower alcohol, such as ethanol, isopropanol, tert-butanol or n-butanol; alkylene polyols, for instance propylene glycol; polyol ethers; and mixtures thereof.

The composition according to the invention may also comprise one or more standard additives that are well known in the art, such as anionic, cationic, nonionic, amphoteric or zwitterionic surfactants, thickeners, nacreous agents, opacifiers, fragrances, mineral or organic, natural or synthetic particles, preserving agents and pH stabilizers.

A person skilled in the art will take care to select the optional additives and the amount thereof such that they do not harm the properties of the compositions of the present invention.

These additives are generally present in the composition according to the invention in an amount ranging from 0 to 20% by weight relative to the total weight of the composition.

The cosmetic compositions in accordance with the invention may be in the form of a mousse, a gel, a spray or a lacquer and may be used in rinse-out or leave-in application.

The compositions in accordance with the invention may be used as hair products, especially rinse-out or leave-in products, and in particular for washing, caring for and conditioning the hair, holding the hairstyle, and shaping, dyeing, bleaching, permanently reshaping or relaxing the hair.

The compositions of the invention may also be used as care or hygiene products such as protective, treating or care creams for the face, the hands or the body, protective or care body milks, gels or mousses for caring for or cleansing the skin, or alternatively as products for making up or for removing makeup from the skin, the lips, the nails and the eyelashes.

The examples below are given as illustrations of the invention.

### EXAMPLE 1

An amphoteric polysaccharide containing a cationic polymer chain is prepared by modifying a sodium carboxymethylcellulose salt by grafting with polymerized diallyldimethylammonium chloride.

9 g of a sodium carboxymethylcellulose salt (low viscosity 500-2500 mPa·s) are dissolved in 300 ml of distilled water, and 4.93 mmol of KMnO₄ are then added.

The mixture is stirred for 30 minutes, and 4.93 mmol of H₂SO₄ and 0.135 mol of diallyldimethylammonium chloride are then added.

A stream of nitrogen is established and the mixture is maintained at 60° C. for 4 hours.

The medium gradually decolorizes. The product is precipitated from 1 litre of isopropanol, isolated by filtration and washed with an ethanol/acetone mixture.

It is dried under vacuum at 40° C. to give 14 g of product.

### EXAMPLE 2

### Shampoo

A shampoo was prepared using the following ingredients, the amounts of which are given as weight percentages of active material relative to the total weight of the composition:

| | |
|---|---|
| Sodium lauryl ether sulfate (Texapon N702 from Cognis) | 12.5% |
| Cocoylbetaïne (Dehyton AB 30 from Goldschmidt) | 2.5% |
| Dimethicone (DC200 Fluid from Dow Corning) | 2.0% |
| Compound of Example 1 | 0.5% |
| Cocamide monoisopropanolamine | 0.4% |
| Carbomer | 0.2% |
| Preserving agent | qs |
| Fragrance | qs |
| Citric acid/sodium hydroxide qs | pH 6.5 |
| Water qs | 100 |

### EXAMPLE 3

### Conditioner

A conditioner was prepared using the following ingredients, the amounts of which are given as weight percentages of active material relative to the total weight of the composition:

| | |
|---|---|
| Behenyltrimethylammonium chloride (Genamin KDMP | 1.2% |
| from Clariant) | |
| PEG/PPG Dimethicone (Abil B8851 from Goldschmidt) | 0.5% |
| Cyclopentasiloxane (Dow Corning 245 Fluid) | 15.0% |
| Compound of Example 1 | 1.0% |
| Propylene glycol | 2.5% |
| Preserving agent | qs |
| Fragrance | qs |
| Citric acid/sodium hydroxide qs | pH 6.5 |
| Water qs | 100 |

### EXAMPLES 4-6

Dye compositions were prepared using the following ingredients, the amounts of which are given as weight percentages of active material relative to the total weight of the composition:

| | Ex. 4 | Ex. 5 | Ex. 6 |
|---|---|---|---|
| para-Phenylenediamine | 0.24 | 0.24 | 0.24 |
| para-Aminophenol | 0.44 | 0.44 | 0.44 |
| 2-Aminophenol | 0.028 | 0.028 | 0.028 |
| 1,3-Dihydroxybenzene | 0.192 | 0.192 | 0.192 |
| 3-Aminophenol | 0.019 | 0.019 | 0.019 |
| 5-N-(β-Hydroxyethyl)amino-2-methylphenol | 0.021 | 0.021 | 0.021 |
| 1,3-Dihydroxy-2-methylbenzene | 0.055 | 0.055 | 0.055 |
| Anhydrous sodium metasilicate | 2 | 2 | 2 |
| Monoethanolamine | 5.45 | 5.45 | 5.45 |
| Reducing agent, antioxidant, sequestering | qs | qs | qs |
| agent, fragrance | | | |
| Propylene glycol | 10 | 10 | 10 |
| Crosslinked acrylic acid polymer | 0.4 | 0.4 | 0.4 |
| Compound of Example 1 | 1.5 | 1.5 | 2.8 |
| Cationic polymer: hexadimethrine chloride | 3 | 3 | — |
| (CTFA name) Mexomer PO sold by the | | | |
| company Chimex | | | |
| Powdered sodium lauryl sulfate | 3 | — | — |
| Lauryl alcohol oxyethylenated with 12 mol | — | 7.5 | 7.5 |
| of ethylene oxide | | | |
| Oleocetyl alcohol oxyethylenated with 30 | — | 4 | 4 |
| mol of ethylene oxide | | | |
| Decyl alcohol oxyethylenated with 3 mol of | 10 | 10 | 10 |
| ethylene oxide | | | |
| Decyl alcohol oxyethylenated with 5 mol of | 8 | — | — |
| ethylene oxide | | | |
| Lauric acid | 2.5 | 2.5 | 2.5 |
| 50/50 cetylstearyl alcohol | 11.5 | 11.5 | 11.5 |
| Nacreous agent: hydrophobic fumed silica | 1.2 | 1.2 | 1.2 |
| Nacreous agent: glyceryl monostearate | 2 | 2 | 2 |
| Demineralized water qs | 100 | 100 | 100 |

At the time of use, each dye composition described above was mixed weight-for-weight with a 20-volumes hydrogen peroxide solution (6% by weight).

The mixtures thus prepared were applied for 30 minutes to locks of natural or permanent-waved grey hair containing 90% white hairs. The locks were then rinsed, washed with a standard shampoo, rinsed again and then dried.

The hair was dyed in a golden-blond shade for each of the Examples 4 to 6.

### EXAMPLE 7

Another dye composition was prepared using the following ingredients, the amounts of which are given as weight percentages of active material relative to the total weight of the composition:

| | |
|---|---|
| Mixture of C18 to C24 linear alcohols | 3 |
| (C18/C20/C22/C24: 7/57/30/6 - alcohol content > 95%) | |
| Oxyethylenated stearyl alcohol (2 mol of ethylene oxide) | 4.5 |
| Oxyethylenated stearyl alcohol (21 mol of ethylene oxide) | 1.75 |
| Oleic acid | 2.6 |
| Cationic polyurethane obtained by condensation of 1,3-bis- | 0.2 |
| (isocyanatomethylcyclohexane), N,N-dimethylethanolamine | |
| quaternized with bromododecane, N,N-dimethylethanolamine | |
| and polyoxyethylene of molecular weight 10 000 | |
| Crosslinked poly(acrylic acid) (product sold under the name | 0.4 |
| Carbopol 980 by the company Noveon) | |
| Hydroxypropylmethylcellulose | 0.2 |
| Coconut acid monoisopropanolamide | 3 |
| Merquat 100 as an aqueous 40% solution | 1.6 |
| Compound of Example 1 | 2 |
| Sodium metabisulfite | 0.71 |
| EDTA (ethylenediaminetetraacetic acid) | 0.2 |
| tert-Butylhydroquinone | 0.3 |
| 1,4-Diaminobenzene | 0.2 |
| para-Aminophenol | 1.2 |
| 1,3-Dihydroxybenzene | 0.1 |
| 1-Hydroxy-3-aminobenzene | 0.2 |
| 1-Methyl-2-hydroxy-4-β-hydroxyethylaminobenzene | 0.8 |
| Monoethanolamine | 1 |
| Aqueous ammonia containing 20% NH₃ | 11 |
| Fragrance | qs |
| Demineralized water qs | 100 |

This composition is mixed at the time of use with an oxidizing composition in emulsion form containing as oxidizing agent 7.5% hydrogen peroxide, in a proportion of 1 part by weight of dye composition per 1.5 parts by weight of oxidizing composition. The mixture obtained is applied to locks of natural hair containing 90% white hairs, and is left to act for 30 minutes. After rinsing, washing with shampoo and drying, hair dyed in a strong coppery-red light-chestnut shade is obtained.

### EXAMPLE 8

The following compositions were prepared, the percentages indicated being weight percentages relative to the total weight of the composition:

Oxidizing composition:

| | |
|---|---|
| Fatty alcohol | 2.3% |
| Oxyethylenated fatty alcohol | 0.6% |
| Fatty amide | 0.9% |
| Glycerol | 0.5% |
| Hydrogen peroxide | 7.5% |
| Fragrance | qs |
| Demineralized water qs | 100% |

Dye composition:

| | |
|---|---|
| Mixture of C18 to C24 linear alcohols | 3% |
| [C18/C20/C22/C24, 7/58/30/6, alcohol content > | |
| 95%] (Nafol 20-22) | |
| Mixture of C18 to C24 linear alcohols | 1.35% |
| [C18/C20/C22/C24, 7/58/30/6, alcohol content > | |
| 95%] in oxyethylenated form (30 mol of ethylene | |
| oxide) (Nafolox 20-22) | |
| Oxyethylenated stearyl alcohol (2 mol of ethylene | 4% |
| oxide) | |
| Oxyethylenated stearyl alcohol (21 mol of ethylene | 2% |
| oxide) | |
| Oleic acid | 2.6% |
| Glycol distearate | 2% |
| Propylene glycol | 5% |
| Coconut acid monoisopropanolamide | 2% |
| Aculyn 44 sold by the company Rohm & Haas | 1.4% AM* |
| Crosslinked poly(acrylic acid) | 0.6% |
| Compound of Example 1 | 3% AM* |
| Merquat 100 sold by the company Calgon | 0.4% AM* |
| Reducing agents | 0.7% |
| Sequestering agents | 0.2% |
| 1,3-Dihydroxybenzene (resorcinol) | 0.6% |
| 1,4-Diaminobenzene | 0.5% |
| 1-Hydroxy-3-aminobenzene | 0.1% |
| 1-Hydroxy-2-aminobenzene | 0.05% |
| 1-Hydroxy-4-aminobenzene | 0.09% |
| 6-Hydroxybenzomorpholine | 0.017% |
| 1-β-Hydroxyethyloxy-2,4-diaminobenzene | 0.039% |
| dihydrochloride | |
| Propylene glycol monobutyl ether | 2.5% |
| Pure monoethanolamine | 1.06% |
| Aqueous ammonia (containing 20.5% ammonia) | 11.1% |
| Water qs | 100% |

AM* = Active Material

The dye composition was mixed, at the time of use, in a plastic bowl and for 2 minutes, with the oxidizing composition given above, in a proportion of 1 part of dye composition per 1.5 parts of oxidizing composition.
The mixture obtained was applied to locks of natural hair containing 90% white hairs, and was left to act for 30 minutes.
The locks were then rinsed with water, washed with shampoo, rinsed again with water and then dried and disentangled.

The hair was then dyed in a strong light-chestnut shade.

### EXAMPLE 9

Another dye composition was prepared using the following ingredients, the amounts of which are given as weight percentages of active material relative to the total weight of the composition:

| | |
|---|---|
| Oleocetyl alcohol oxyethylenated with 30 mol of | 7% |
| ethylene oxide (nCA = 17 - HLB = 16.5) | |
| Lauryl alcohol (C12-C14/55-45%) oxyethylenated with | 8% |
| 12 mol of ethylene oxide (nCA = 12.5 - HLB = 14) | |
| Cetylstearyl alcohol (C16/C18-50/50)(nCB = 17- | 5% |
| HLB = 1) | |
| Decyl alcohol (C10-C12-C14/85-8.5-6.5) oxyethylenated | 22% |
| with 3.5 mol of ethylene oxide, sold under the name | |
| Mergital BL 309 by the company Henkel (nCB = 10.4 - | |
| HLB = 8.5) | |
| Copolymer of diallyldimethylammonium chloride and of | 3% AM |
| acrylic acid, sold under the name Merquat 280 by the | |
| company Calgon, containing 35% AM | |
| Compound of Example 1 | 1% |
| Crosslinked poly(acrylic acid) sold under the name | 0.4% |
| Carbopol 934 (MW 3 000 000) by the company Goodrich | |
| Propylene glycol | 8% |
| Monoethanolamine | 8.3% |
| Hydroquinone | 0.1% |
| 1-Phenyl-3-methyl-5-pyrazolone | 0.1% |
| Aqueous sodium bisulfite solution containing 35% AM | 1.3% |
| para-Phenylenediamine | 0.5% |
| m-Dihydroxybenzene | 0.4% |
| Fragrance, sequestering agent | qs |
| Eau qs | 100% |
| pH = 11.0 | |

AM: Active Material

1-20. (canceled)

21. A method for the cosmetic treatment of keratin materials, comprising applying to said keratin materials at least one amphoteric polysaccharide compound comprising at least one cationic polymer chain, wherein said at least one polysaccharide compound may be obtained by grafting and polymerization of ethylenic monomers of formula (I): 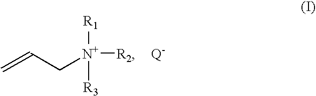
onto anionic polysaccharides of formula (II): 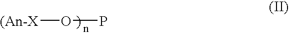
in the presence of a catalytic system comprising potassium permanganate (KMnO₄) and sulfuric acid (H₂SO₄),
wherein:
R₁, R₂ and R₃, which may be identical or different, are each independently chosen from hydrogen and linear or branched, saturated or unsaturated C₁-C₂₂ monovalent hydrocarbon-based groups;
Q is a mineral or organic anion,
P is a polysaccharide chain,
X is a linear or branched, saturated or unsaturated, optionally hydroxylated C₁-C₁₈ divalent hydrocarbon-based group,
An is an ionic group chosen from: 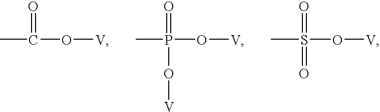
wherein
V is independently chosen from a hydrogen atom, an alkali metal, and an alkaline-earth metal, and
n is chosen such that the degree of substitution of the polysaccharide compound with an anionic group (DS(−)), ranges from 0.02 to 2.

22. The method of claim 21, wherein P is chosen from a cellulose, a hemicellulose, a starch, an insulin, a guar gum, a xanthan gum, a pullulan, an agar-agar, a sodium, potassium or ammonium alginate, a carrageenan, a dextran, a furcellaran, a gellan gum, a gum arabic, a gum tragacanth, a hyaluronic acid, a konjac mannan, a lignin sulfonate, a carob gum, a partially N-acetylated chitin, a pectin, a polydextrose, a rhamsan gum and a welan gum.

23. The method of claim 22, wherein P is chosen from a cellulose, a hemicellulose, a hydroxyethylcellulose, a hydroxypropylcellulose, a hydroxypropylmethylcellulose, a methylcellulose, a starch, a starch acetate, a hydroxyethyl starch, a hydroxypropyl starch, an insulin, a guar gum, a hydroxyethylguar gum, a hydroxypropylguar gum and a xanthan gum.

24. The method of claim 21, wherein said linear or branched, saturated or unsaturated C₁-C₂₂ monovalent hydrocarbon-based group is chosen from a linear or branched C₁-C₄ alkyl group, a C₁₂-C₁₈ linear or branched alkyl group, and a linear or branched C₂-C₆ alkenyl group.

25. The method of claim 24, wherein the linear or branched, saturated or unsaturated C₁-C₂₂ monovalent hydrocarbon-based group is chosen from methyl, ethyl, n-propyl, isopropyl, n-butyl, tert-butyl, lauryl, myristyl, cetyl or stearyl, vinyl, allyl, crotonyl and butenyl groups.

26. The method of claim 21, wherein the linear or branched, saturated or unsaturated C₁-C₁₈ divalent hydrocarbon-based group X is chosen from linear or branched C₁-C₁₈ alkylene groups and linear or branched C₂-C₁₈ alkenylene groups, wherein said groups are optionally substituted with at least one hydroxyl group.

27. The method of claim 26, wherein the linear or branched, saturated or unsaturated C₁-C₁₈ divalent hydrocarbon-based group X is chosen from methylene, ethylene, n-propylene, isopropylene, n-butylene, tert-butylene, hexylene, octylene, vinylene, allylene, crotonylene, isobutenylene, hexenylene and octenylene groups, wherein said groups are optionally substituted with at least one hydroxyl group.

28. The method according to claim 21, wherein Q is chosen from a halogen atom, an acetate, a citrate, a lactate, an oleate and a behenate.

29. The method of claim 28, wherein Q is a halogen atom.

30. A method according to claim 21, wherein said cosmetic treatment is chosen from caring for and protecting the hair, styling and hold of the hair, and cleansing and caring for the skin.

31. A cosmetic composition comprising, in a cosmetically acceptable medium,
at least one cosmetic active agent and
at least one amphoteric polysaccharide compound comprising at least one cationic polymer chain, wherein said at least one polysaccharide compound may be obtained by grafting and polymerization of ethylenic monomers of formula (I): 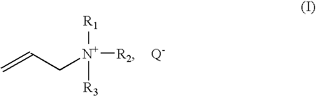
onto anionic polysaccharides of formula (II): 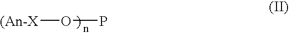
in the presence of a catalytic system comprising potassium permanganate (KMnO₄) and sulfuric acid (H₂SO₄),
wherein:
R₁, R₂ and R₃, which may be identical or different, are each independently chosen from hydrogen and linear or branched, saturated or unsaturated C₁-C₂₂ monovalent hydrocarbon-based groups;
Q is a mineral or organic anion,
P is a polysaccharide chain,
X is a linear or branched, saturated or unsaturated, optionally hydroxylated C₁-C₁₈ divalent hydrocarbon-based group,
An is an ionic group chosen from: 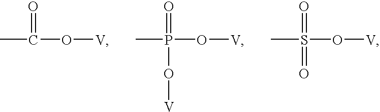
wherein
V is independently chosen from a hydrogen atom, an alkali metal, and an alkaline-earth metal, and
n is chosen such that the degree of substitution of the polysaccharide compound with an anionic group (DS(−)), ranges from 0.02 to 2.

32. A cosmetic composition according to claim 31, wherein said at least one cosmetic active agent is chosen from conditioning agents, dyes, anti-UV agents, sunscreens, hydroxy acids and vitamins.

33. The cosmetic composition of claim 31, wherein said amphoteric polysaccharide comprises an aldehyde function in an amount ranging from 0.05% to 50% by weight, relative to the total weight of the composition.

34. The cosmetic composition of claim 33, wherein said amphoteric polysaccharide comprises an aldehyde function in an amount ranging from 0.5% to 25% by weight, relative to the total weight of the composition.

35. The cosmetic composition of claim 31, wherein said cosmetically acceptable medium comprises water or a mixture of water and of at least one organic solvent.

36. The cosmetic composition of claim 35, wherein the at leas tone organic solvent is chosen from C₁-C₄ lower alcohols, alkylene polyols, polyol ethers, and mixtures thereof.

37. The cosmetic composition of claim 31, further comprising at least one additive chosen from anionic, cationic, nonionic, amphoteric or zwitterionic surfactants, thickeners, nacreous agents, opacifiers, fragrances, mineral or organic, natural or synthetic particles, preserving agents and pH stabilizers.

38. The cosmetic composition of claim 31, wherein said cosmetic composition is in a from chosen from a fixing agent for hair, a conditioning agent for hair and a cleansing and/or care agent for the skin.

39. A method for the cosmetic treatment of keratin materials, comprising applying to said keratin materials a cosmetic composition comprising, in a cosmetically acceptable medium,
at least one cosmetic active agent and
at least one amphoteric polysaccharide compound comprising at least one cationic polymer chain, wherein said at least one polysaccharide compound may be obtained by grafting and polymerization of ethylenic monomers of formula (I): 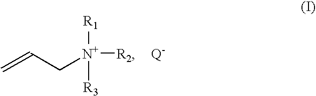
onto anionic polysaccharides of formula (II): 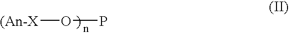
in the presence of a catalytic system comprising potassium permanganate (KMnO₄) and sulfuric acid (H₂SO₄),
wherein:
R₁, R₂ and R₃, which may be identical or different, are each independently chosen from hydrogen and linear or branched, saturated or unsaturated C₁-C₂₂ monovalent hydrocarbon-based groups;
Q is a mineral or organic anion,
P is a polysaccharide chain,
X is a linear or branched, saturated or unsaturated, optionally hydroxylated C₁-C₁₈ divalent hydrocarbon-based group,
An is an ionic group chosen from: 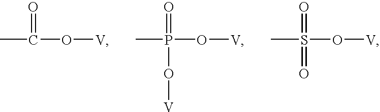
wherein
V is independently chosen from a hydrogen atom, an alkali metal, and an alkaline-earth metal, and
n is chosen such that the degree of substitution of the polysaccharide compound with an anionic group (DS(−)), ranges from 0.02 to 2.
